(19) United States
(12) Patent Application Publication
(10) Pub. No.: US 2008/0113915 A1
(43) Pub. Date: May 15, 2008

(54) SAPAP3 knockout mouse and clinical modeling associated with the SAPAP3 gene

(75) Inventors: Jeffrey M. Welch, Durham, NC (US); Guoping Feng, Chapel Hill, NC (US)

Correspondence Address: JENKINS, WILSON, TAYLOR & HUNT, P. A., 3100 TOWER BLVD., Suite 1200, DURHAM, NC, 27707, US

(73) Assignee: Duke University, Durham, NC (US)

(21) Appl. No.: 11/973,304

(22) Filed: Oct. 5, 2007

Related U.S. Application Data
(60) Provisional application No. 60/865,108, filed on Nov. 9, 2006.

(51) Int. Cl. A01K 67/027; A61K 38/00; A61P 25/00; C12Q 1/68; G01N 33/00

(52) U.S. Cl. 514/12; 435/6; 800/18; 800/3

(57) ABSTRACT

Provided is a transgenic knockout mouse whose genome includes a disruption in its endogenous SAPAP3 gene, wherein the disruption results in the mouse exhibiting increased levels of anxiety as compared to a wild type mouse. Also provided are cells derived from the disclosed transgenic knockout mouse. Also provided are methods of identifying a therapeutic agent for the treatment of an individual diagnosed with anxiety, methods for diagnosing an individual with a clinical disorder associated with reduced expression of a SAPAP3 gene product, and methods for treating an individual with a clinical anxiety disorder associated with reduced expression of a SAPAP3 gene product.

### RELATED APPLICATIONS

The presently disclosed subject matter claims the benefit of U.S. Provisional Patent Application Ser. No. 60/865,108, filed Nov. 9, 2006, the disclosure of which is incorporated herein by reference in its entirety.

### GOVERNMENT INTEREST

This presently disclosed subject matter was made with U.S. Government support under Grant Nos. 1 F31 NS050899-01 and R01 NS42609-01A1 from the National Institutes of Health, and Grant No. DGE-0202746 from the National Science Foundation. Thus, the U.S. Government has certain rights in the presently disclosed subject matter.

### TECHNICAL FIELD

The presently disclosed subject matter relates to a transgenic mouse constructed to lack a functional copy of the SAPAP3 gene which has been discovered to be useful in the ability to help diagnose, treat, and discover new pharmacological agents for anxiety related disorders. The presently disclosed subject matter also relates to the SAPAP3 gene, which has been discovered to be useful in the ability to help diagnose, treat, and discover new pharmacological agents for anxiety related disorders

### BACKGROUND

The SAPAP proteins have been known since the mid 1990's due to interactions measured with the GK domains of SAP90/PSD-95 and hDIg/SAP97. The SAPAP family consists of four genes, SAPAP 1 through 4, each exhibiting roughly 50% identity at the amino acid level. Original studies suggested that the expressed proteins might act as linkers between certain ion channel/receptors and the cytoskeleton. However more recent findings indicate that these proteins can play a role in intracellular signaling. Recent studies have also indicated that there is a possible role for these proteins in synaptic plasticity. Only recently have the protein localizations of the SAPAP proteins been studied. The SAPAPs are expressed in the nervous system and are located predominantly at synapses. The proteins not only are found to have glutamatergic localization they also are found at cholinergic synapses (Welch et al. J Comp Neur 472:24-39, 2004).

It has been discovered that SAPAP3 is highly expressed in several regions of the brain, with particular note of high expression in the striatum for purposes of the presently disclosed subject matter. It is also important to note that the other SAPAP family members (1, 2, and 4) are not expressed or are minimally expressed in the striatum. It was also found that SAPAP3 mRNA was localized in dendrites and in layer one of the cerebral cortex (Welch et al. J Comp Neur 472:24-39, 2004).

To date, the role of the SAPAP3 protein in anxiety or the value of the protein and gene has not been established. Anxiety disorder has been defined as excessive or inappropriate feelings of apprehension, uncertainty, and fear. Often, the feelings have no real basis to which the feelings can be attributed. Nevertheless, an anxiety disorder can cause an individual to withdraw or to be incapable of action. Anxiety can also be a symptom of other psychological or medical problems, such as depression, substance abuse, self-image problems, or thyroid disease. Anxiety disorders are the most common diagnosed psychiatric condition in the United States. It appears that about 25 million Americans experience some form of an anxiety disorder at some time during their lives. In recent years, a number of different anxiety disorders have been classified. Anxiety disorders will fall into two categories: DSM-IV anxiety class disorders and disorders co-morbid with Obsessive Compulsive Disorder (OCD; a specific DSM-IV disorder). Examples of DSM-IV Anxiety disorders include: obsessive compulsive disorder (CD), Generalized Anxiety disorder, acute stress disorder, and posttraumatic stress disorder, social and specific phobias, agoraphobia, panic attack, and the like. Disorders which are generally recognized as co-morbid with OCD include: major depression, simple phobia, eating disorders, alcohol abuse, panic disorder, Tourette's syndrome, trichotillomania, and the like.

Although a number of different anti-anxiolytics such as benzodiazepines and serotonin selective reuptake inhibitors (SSRIs) have been developed for use in the treatment of anxiety disorders, there is a continued interest in the development of new treatment methodologies and agents for use in treating these conditions.

### SUMMARY

The presently disclosed subject matter relates in some embodiments to the SAPAP3 gene, SAPAP3 gene product, and SAPAP3 deficient mice that have been discovered to be useful in testing compositions for anti anxiety properties. The presently disclosed subject matter also relates in some embodiments to methods of testing compositions for use in treating anxiety and the treatment of SAPAP3 related anxiety.

The presently disclosed subject matter also relates in some embodiments to a SAPAP3 knockout mouse. In particular, the presently disclosed subject matter relates to transgenic knockout mouse whose genome comprises a disruption in its endogenous SAPAP3 gene wherein the disruption results in the mouse exhibiting increased levels of anxiety as compared to a wild type mouse.

The presently disclosed subject matter provides methods for identifying a therapeutic agent for the treatment of an individual diagnosed with an anxiety disorder in the individual. In some embodiments, the methods comprise (a) providing a transgenic knockout mouse whose genome comprises a disruption in its endogenous SAPAP3 gene wherein the disruption results in the mouse exhibiting increased levels of anxiety as compared to a wild type mouse; (b) administering a candidate therapeutic agent to the mouse of step (a); and (c) assaying the therapeutic effects of the candidate therapeutic agent by comparing the symptoms of the anxiety disorder in the knockout mouse which has received the candidate agent of step (b) with the symptoms of a transgenic knockout mouse of step (a) which has not received the candidate therapeutic agent, wherein amelioration of one or more of the symptoms of the anxiety disorder in the mouse of step (b) is an indication of the therapeutic effect of the candidate therapeutic agent.

In some embodiments, the presently disclosed methods comprise (a) providing a mammalian cell comprising a disruption in an endogenous SAPAP3 gene, wherein the disruption results in a reduced level of an SAPAP3 biological activity in the mammalian cell as compared to that seen in a wild type cell under identical conditions; (b) administering a test compound to the cell of step (a); and (c) assaying the therapeutic effects of the test compound by comparing the level of an SAPAP3 biological activity in the cell after the administering step with the level of an SAPAP3 biological activity in the cell prior to the administering step, wherein an increase in the level of an SAPAP3 biological activity in the cell after the administering step is an indication that the test compound is a therapeutic agent for the treatment of anxiety. In some embodiments, the mammalian cell is present within a transgenic non-human mammal. In some embodiments, the administering results in an upregulation in expression of an endogenous SAPAP3 allele sufficient to result in an increased level of an SAPAP3 biological activity in the cell after the administering step.

In some embodiments, the presently disclosed methods comprise (a) providing a non-human mammal that comprises a disruption of one or both SAPAP3 alleles, wherein the non-human mammal expresses an anxiety phenotype; (b) administering a test compound to the non-human mammal; and (c) comparing an anxiety phenotype in the non-human mammal before administering the test compound to an anxiety phenotype in the non-human mammal after administering the test compound, whereby a therapeutic agent for the treatment of anxiety in a subject is identified.

The presently disclosed subject matter provides methods for identifying a therapeutic agent for the treatment of an individual diagnosed with an anxiety disorder associated with a reduction of expression of wild type SAPAP3 protein in the individual. In some embodiments, the methods comprise (a) providing a mammalian cell whose genome comprises a disruption in its endogenous SAPAP3 gene wherein the disruption results in a reduced expression of wild type SAPAP3 protein as compared to a wild type mouse; (b) administering a candidate therapeutic agent to the cell of step (a); and (c) assaying the therapeutic effects of the candidate therapeutic agent by comparing the amount of SAPAP3 produced in the cell which has received the candidate agent of step b) with the amount of SAPAP3 produced by a cell of step (a) which has not received the candidate therapeutic agent, wherein an increase in the amount of SAPAP3 protein produced by the cell of step (b) is an indication of the therapeutic effect of the candidate therapeutic agent.

The presently disclosed subject matter also provides methods for diagnosing an individual with a clinical disorder associated with reduced expression of SAPAP3. In some embodiments, the methods comprise (a) providing a tissue sample from the individual; (b) quantitatively detecting the expression of SAPAP3 protein in cells of the sample; and (c) assaying the amount of SAPAP3 protein produced by comparing the quantity of SAPAP3 protein produced in step b) with the quantity of SAPAP3 protein produced by cells known to exhibit the normal expression of SAPAP3 wherein a substantial decrease in SAPAP3 protein in the sample by comparison is indicative of the clinical disorder in the individual.

The presently disclosed subject matter also provides methods for treating an individual with a clinical anxiety disorder associated with a reduced level of a biological activity of an SAPAP3 gene product in a biological sample. In some embodiments, the methods comprise (a) assaying a biological sample isolated from the individual to determine that the individual has a reduced level of a biological activity of an SAPAP3 gene product in the biological sample compared to the biological sample isolated from a control individual; and (b) administering to the individual an effective amount of an enhancer of a biological activity of an SAPAP3 gene product sufficient to ameliorate the clinical anxiety disorder. In still another embodiment, the biological sample is selected from the group consisting of a cell, a tissue, and a fluid that normally would be expected to express an SAPAP3 gene product in an individual of the same species as the individual from which the biological sample was isolated. In some embodiments, the individual is a mammal. In some embodiments, the administering comprises introducing into a cell or a tissue of the individual an expression construct encoding an SAPAP3 gene product operably linked to a promoter that is active in the cell or tissue, whereby an amount of an SAPAP3 protein sufficient to ameliorate the clinical anxiety disorder is produced in the cell of the tissue. In some embodiments, the enhancer of a biological activity of an SAPAP3 gene product comprises a purified SAPAP3 protein and the administering comprises administering to the individual an amount of the purified SAPAP3 protein sufficient to ameliorate the clinical anxiety disorder to the individual.

The presently disclosed subject matter also provides methods for treating an individual with a clinical disorder associated with reduced expression of SAPAP3 protein caused by a genetic mutation in the SAPAP3 gene. In some embodiments, the methods comprise (a) providing an expression vector containing nucleic acid encoding the SAPAP3 gene of the wild type form without the mutation; and (b) introducing the vector into the individual under conditions appropriate for expression of the wild type for of the SAPAP3 protein.

The presently disclosed subject matter also provides cell derived and/or isolated from the disclosed transgenic non-human mammals, wherein the cell comprises a disruption in its endogenous SAPAP3 gene. In some embodiments, the cell is a somatic cell, and in some embodiments the cell is a germ cell. In some embodiments, the cell is an embryonic stem (ES) cell. In some embodiments, the ES cell comprises a hemizygous or heterozygous disruption of one SAPAP3 allele, and in some embodiments the ES cell is homozygous for an SAPAP3 disruption.

It is an object of the presently disclosed subject matter to provide a SAPAP3 knockout mouse.

An object of the presently disclosed subject matter having been stated hereinabove, and which is achieved in whole or in part by the presently disclosed subject matter, other objects will become evident as the description proceeds when taken in connection with the accompanying drawings as best described hereinbelow.

### BRIEF DESCRIPTION OF THE FIGURES

FIG. 1 is a schematic of the SAPAP3 gene, the targeting vector, and the homologous recombinant gene.

FIG. 2 is a Western blot for SAPAP3 protein in post-synaptic density fractions prepared from two SAPAP3 knockout mice and two wild type mice.

FIG. 3 is a bar graph comparing cohorts of SAPAP3 knockout mice and wild type mice of the average number of bouts of grooming engaged in during videotaping sessions.

FIG. 4 is a bar graph comparing cohorts of SAPAP3 knockout mice and wild type mice of the average amount of time spent grooming during videotaping sessions.

FIG. 5 is a bar graph comparing pre-lesion and post-lesion groups of SAPAP3 knockouts for average number of bouts of grooming engaged in during videotaping sessions, analyzed as in FIG. 3.

FIG. 6 is a line graph comparing cohorts of SAPAP3 knockout mice and wild type mice of the average time spent in the “center” of an open field, as binned in 5 minute intervals across a 30 minute test.

FIG. 7 is a bar graph comparing cohorts of SAPAP3 knockout mice and wild type mice of the average time spent in the “center” of an open field averaged across 30 minutes of testing.

FIG. 8 is a bar graph comparing cohorts of SAPAP3 knockout and wild type mice of the average latency to leave a dark chamber to explore a brightly-lit chamber.

FIG. 9 is a bar graph comparing cohorts of SAPAP3 knockout and wild type mice of the average amount of time spent in the brightly lit chamber vs. the dark chamber over 5 minutes of testing.

FIG. 10 is a bar graph comparing a cohort of SAPAP3 mice dosed intraperitoneally with 5 mg/kg Fluoxetine and a cohort of SAPAP3 mice dosed with vehicle for average number of bouts of grooming engaged in during videotaping sessions, analyzed as in FIG. 3.

FIG. 11 is a bar graph comparing a cohort of SAPAP3 mice dosed intraperitoneally with 5 mg/kg Fluoxetine and a cohort of SAPAP3 mice dosed with vehicle of the average latency to leave a dark chamber to explore a brightly-lit chamber, analyzed as in FIG. 8.

FIG. 12 is a line graph comparing average N2 trace size in electrophysiological recordings of cortico-striatal synapse response. Inset is one raw electrophysiological trace showing the definition of the N2 synaptic event.

FIG. 13 is raw Western blot data comparing protein levels between post-synaptic density fractions simultaneously prepared from one SAPAP3 knockout out one wild type mouse.

FIG. 14 is a bar graph comparing average ratios of protein in 3 pairs of post-synaptic density fractions prepared simultaneously from SAPAP3 knockout and wild type mice as in FIG. 13. Data are normalized to levels of beta-tubulin.

FIG. 15 is raw data collected from cortico-striatal slices from both SAPAP3 knockout and wild type mice as in FIG. 12, but with AMPA (alpha amino propionic acid) receptors blocked, thereby exposing NMDA (N-methyl-D-aspartate) receptor-mediated currents.

FIG. 16 is a bar graph comparing SAPAP3 knockout and wild type mice of the average NMDA current from cortico-striatal slices, collected as in FIG. 15.

### DETAILED DESCRIPTION

The above interests in developing further anti-anxiety compositions and more, as can readily be seen from the disclosure which follows, are addressed by the presently disclosed subject matter. This detailed description defines the meaning of the terms used herein and specifically describes embodiments in order for those skilled in the art to practice the presently disclosed subject matter.

While the following terms are believed to be well understood by one of ordinary skill in the art, the following definitions are set forth to facilitate explanation of the presently disclosed subject matter.

All references listed in the instant disclosure, including but not limited to all patents, patent applications and publications thereof, scientific journal articles, and database entries (including GENBANK® database entries and all annotations available therein) are incorporated herein by reference in their entireties to the extent that they supplement, explain, provide a background for, or teach methodology, techniques, and/or compositions employed herein.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one of skill in the art. While the following terms are believed to be well understood by one of ordinary skill in the art, the following definitions are set forth to facilitate explanation of the presently disclosed subject matter.

Following long-standing patent law convention, the terms “a”, “an”, and “the” mean “one or more” when used in this application, including the claims. Thus, the phrase “an apoptotic cell associated with a responding tumor” refers to one or more apoptotic cells associated with one or more responding tumors.

As used herein, the term “SAPAP3 gene” refers to a gene that encodes an SAP90/PSD95-associated protein 3 homolog or ortholog from a vertebrate or invertebrate species. SAPAP3 genes are also known by the gene name “disks large-associated protein 3”. Representative SAPAP3 genes and gene products are disclosed in the GENBANK® database under the following Accession Nos.: from Mus musculus, NM_{—}198618 (nucleotide) and_NP_{—}941020 (amino acid), respectively; from Homo sapiens, NM_{—}001080418 (nucleotide) and NP_{—}001073887 (amino acid), respectively; and from Rattus norvegicus, NM_{—}173138 (nucleotide) and NP_{—}775161 (amino acid), respectively.

As used herein, the term “SAPAP3 biological activity” refers to any naturally occurring biological activity that an SAPAP3 gene product has in vivo. Exemplary SAPAP3 biological activities include, but are not limited to interacting with members of the MAGUK (membrane associated guanylate kinase) family (e.g., PSD-95), the SHANK family, kinases, kinase binding proteins, and others.

Additional biological activities include those that SAPAP3 has in modulating the activity of cortico-striatal synapses.

As used a “knockout mouse” is a mouse that contains within its genome a specific gene that has been inactivated by disruption, for example, by deletion or gene targeting. A knockout mouse includes both the heterozygote mouse (having one disrupted allele and one undisrupted allele) and the homozygous mutant having two defective alleles. Gene targeting is a type of gene recombination that occurs when a fragment of genomic DNA is introduced into a cell such that that fragment locates and recombines with endogenous homologous sequences. Such recombination can be as a replacement for deleted sequences but also can be an insertion of additional DNA. One important example of such insertion is the use of antibiotic resistance genes, such as the Neomycin/G418 resistance gene. Antibiotic resistance genes are well known and useful in helping select for modified cells with the appropriate insertion. A further example of deletion in the SAPAP3 gene is the deletion of one or more of the exons of the SAPAP3 gene. In an exemplary embodiment detailed in the examples the second and third exon are deleted.

Gene deletion can result from a deletion of a portion of an endogenous gene or deletion of the complete gene. These substitutions and deletions can cause the protein encoded for by the SAPAP3 gene to either be expressed incorrectly (and thus not function properly) or to not be expressed at all. The knockout mouse of the presently disclosed subject matter exhibits one or more symptoms of anxiety. In the examples below, an embodiment of the mice of the presently disclosed subject matter includes such symptoms as excessive grooming indicating an OCD and bright light aversion indicating change anxiety.

As used herein “anxiety” refers to a mouse or other mammalian subject for example a human, suffering unwanted anxiety, excessive anxiety, anxiety disorders, and the like as described above. Similarly, the term “anxiety phenotype” refers to any visual or otherwise detectable manifestation of anxiety. Exemplary anxiety phenotypes include, but are not limited to apprehension of danger and or dread accompanied by one or more of restlessness, tension, tachycardia and dyspnea wherein the apprehension might or might not be attached to a clearly identifiable stimulus. In some embodiments, an anxiety phenotype comprises excessive grooming in mice, optionally leading to lesion formation. As used herein, the term “anxiety” also includes DSM-IV anxiety class disorders and disorders co-morbid with OCD.

As used herein “exhibiting increased levels of anxiety as compared to wild type mouse” refers to an observable or measurable difference in the anxiety symptoms in the knockout mouse versus a wild type mouse. For example, the examples herein indicated that the SAPAP3 knockout mouse of the presently disclosed subject matter exhibits excessive grooming habits as well as increased anxiety relating to novel situations or stimuli.

As used herein “cell derived from a knockout mouse” refers to cells either in the mouse itself or separated from the mouse and still living and expressing the gene mutation as described herein. The cell can be useful by itself for cell based assays using the mutated gene or can likewise just refer to individual cells in the knockout mouse.

As used herein “therapeutic agent” refers to a test compound, as defined herein, which has been shown to ameliorate the symptoms of anxiety in a test subject. A test compound can be tested on a subject with anxiety and then compared to another subject without anxiety or compared to the subject before treatment. Where the test compound ameliorates the anxiety symptoms in the test subject the test compound can be considered a therapeutic agent. A test compound can also be considered a therapeutic agent where increases in the amount of SAPAP3 protein are produced in an individual mammal.

As used herein “treatment” refers to at least an amelioration of the symptoms associated with the anxiety affecting the host, where amelioration is used in a broad sense to refer to at least a reduction in the magnitude of a parameter, e.g. symptom, such as level or magnitude of apprehension, uncertainty, and fear, etc. As such, treatment also includes situations where the pathological condition, or at least symptoms associated therewith, are completely inhibited, e.g. prevented from happening, or stopped, e.g. terminated, such that the host no longer suffers from the condition, or at least the symptoms that characterize the condition.

A variety of hosts are treatable according to the methods of the presently disclosed subject matter. Generally, such hosts are “mammals” or “mammalian,” where these terms are used broadly to describe organisms which are within the class Mammalia, including the orders Carnivora (e.g., dogs and cats), Rodentia (e.g., mice, guinea pigs, and rats), and Primates (e.g., humans, chimpanzees, and monkeys). In some embodiments, the hosts are humans.

The production of SAPAP3 knockout mice or other knockout mammal can be carried out in view of the disclosure provided herein and in light of techniques known to those skilled in the art, such as described in U.S. Pat. No. 5,767,337 to Roses et al.; U.S. Pat. No. 5,569,827 to Kessous-Elbaz et al.; and U.S. Pat. No. 5,569,824 to Donehower et al.; U.S. Pat. No. 6,207,878 to Campbell et al., and Harada et al., Nature 369:488, 1994), the disclosure of each of which applicants specifically intend to be incorporated by reference herein in its entirety). Representative embodiments of the presently disclosed subject matter comprising mice for carrying out the presently disclosed subject matter are also disclosed in the examples below. The skilled artisan will be aware of numerous other methods for generating a knockout mouse. The skilled artisan will also realize that a SAPAP3 deficiency can also be produced in other animal species to generate other SAPAP3 protein altered animals. It is the intent of the presently disclosed subject matter to encompass all such equivalents. The presently disclosed subject matter also encompasses all cells, tissues, organs, and progeny derived from the SAPAP3 deficient animal described herein.

The knockout mouse of the presently disclosed subject matter exhibits phenotypic differences when compared to an otherwise genetically identical control mouse containing an uninterrupted SAPAP3 gene. (Comparison of the SAPAP3 deficient mouse to the control mouse was made under conditions wherein all mice were raised and maintained in the same environment under the same conditions). One such difference in phenotype relates to OCD. The knockout mouse of the presently disclosed subject matter exhibits substantially more grooming behavior as compared to the control mouse, when fed identical food and allowed to eat and drink at will. The Exemplification section below details experiments which characterize the knockout mouse as grooming to the point where lesions appear on the mouse.

Another phenotype of the knockout mice relates to anxiety behavior in a light dark test or an open field test. The experiments detailed below indicate that measurable anxiety disorder exists in the knockout mouse. The experiments below also show that when treated with a known anti-anxiolytic compound, such as Fluoxetine, that anxious behavior, grooming bouts as well as reduction in lesion is reduced over a period of time. Without wishing to be bound by theory, the increased anxiety is thought to reflect a disruption in imbalances in serotonin in the striatum and possibly the frontal cortex and/or reduced overall excitatory input to the striatum from the cortex. It is shown herein that the knockout mice have an overabundance of NMDA receptors in both biochemical and electrophysiological experiments and thus exhibit altered glutamatergic neurotransmission in the striatum.

The phenotype of the knockout mouse of the presently disclosed subject matter indicates that the SAPAP3 protein plays a significant role in either direct or indirect regulation of an animal's ability to regulate anxiety disorders. Without wishing to be bound by theory, the SAPAP3 protein is thought to be involved in transducing/regulating signaling and structural changes at the post-synaptic site. Importantly, results presented in the Exemplification section below indicate that the absence of functional SAPAP3 gene in an individual, such as a human, contributes to one or more clinical disorders. A clinical disorder that is associated with or directly caused by a reduction or absence of wild type SAPAP3 protein in the diagnosed patient is referred to herein as a SAPAP3 disorder. A SAPAP3 disorder can result from aberrant expression of a SAPAP3 gene or aberrant function of an expressed SAPAP3 protein. One of skill in the art would predict from the phenotype of the SAPAP3 knockout mouse that an individual with reduced or absent wild type SAPAP3 is at risk for development of a number of other clinical disorders and complications known to arise from synaptic abnormalities, including all manner of psychiatric clinical disorders without firmly understood origin at the present time, but that are thought to arise through defective synaptic signaling. Causes of a SAPAP3 disorder are not limited to loss or decrease in function of wild type SAPAP3 gene, in that overproduction or over activity of the SAPAP3 gene in an individual is also expected to produce a clinical disorder which manifests itself as abnormal.

A SAPAP3 disorder can arise from a number of potential genetic defects. Aberrant (reduced or increased) expression of wild type SAPAP3, possibly resulting from a mutation in SAPAP3 gene regulatory sequences, or a mutation in a regulatory protein of SAPAP3 gene expression or protein function is expected to lead to a SAPAP3 disorder. Alternatively, expression of a mutant SAPAP3 protein having impaired function would lead to a SAPAP3 disorder. All animals known to express SAPAP3 (e.g., rodents, canines, felines, equines, and primates, especially humans) can potentially have a SAPAP3 disorder, and can be diagnosed as such by the methods described below.

The presently disclosed subject matter can be used to identify test compounds which will be useful as a therapeutic agent for the treatment of an individual diagnosed with anxiety. Test compounds or libraries of test compounds (where groups of different test compounds are employed), are natural or synthetic organic compounds, including but not limited to oligomers, non-oligomers, or combinations thereof. Non-oligomers include a wide variety of organic molecules, such as heterocyclics, aromatics, alicyclics, aliphatics, and combinations thereof, comprising steroids, antibiotics, enzyme inhibitors, ligands, hormones, drugs, alkaloids, opioids, benzodiazepenes, terpenes, porphyrins, toxins, catalysts, as well as combinations thereof. Oligomers include peptides (that is, oligopeptides) and proteins, oligonucleotides (the term oligonucleotide also referred to simply as “nucleotide”, herein) such as DNA and RNA, oligosaccharides, polylipids, polyesters, polyamides, polyurethanes, polyureas, polyethers, poly (phosphorus derivatives) such as phosphates, phosphonates, phosphoramides, phosphonamides, phosphites, phosphinamides, etc., poly (sulfur derivatives) such as sulfones, sulfonates, sulfites, sulfonamides, sulfenamides, etc., where for the phosphorous and sulfur derivatives the indicated heteroatom for the most part will be bonded to C, H, N, O or S, and combinations thereof. Numerous methods of synthesizing or applying such probe molecules on solid supports (where the probe molecule can be either covalently or non-covalently bound to the solid support) are known, and such probe molecules can be made in accordance with procedures known to those skilled in the art. See e.g., U.S. Pat. No. 5,565,324 to Still et al., U.S. Pat. No. 5,284,514 to Ellman et al., U.S. Pat. No. 5,445,934 to Fodor et al., the disclosure of each of which is incorporated herein by reference in its entirety.

In identifying which test compound will be useful as a therapeutic agent for the treatment of anxiety, a candidate test compound is administered to a knockout mouse of the presently disclosed subject matter. The effect of the candidate test compound on the anxiety in the knockout mouse is determined and related to the anxiety in a knockout mouse of the presently disclosed subject matter which has not received a test compound, but which has otherwise been treated identically. This can be done on an individualize mouse or could be done on an average basis to avoid error due to individualized over or under response in a single mouse. In these screening methods, the candidate test compound is administered to the animal and the animal is then subjected to an anxiety measurement assay, e.g. observation of grooming habits or conduct in the light/dark anxiety test, as described in the experimental section supra. The anxiety of the animal in the assay is then determined and related to the anxiolytic activity of the non treated knockout mouse by comparison as a control. Those test compounds which show an amelioration of the anxiety symptoms are identified as a therapeutic agent for the treatment of anxiety.

In another embodiment of the presently disclosed subject matter a therapeutic agent is identified which is useful in the treatment of anxiety. In this embodiment a mammalian cell (which could be as the whole knockout animal or a single cell or any organ or tissues or the like) is provided whose genome comprises a disruption in the endogenous SAPAP3 protein as compared to a wild type cell. A test compound is administered to the cell and the assay consists of comparing the amount of SAPAP3 protein in the cell to which the test compound is administered to a cell which no test compound is administered. An increase in the amount of SAPAP3 protein is in indicator of a test compound which is useful as a therapeutic agent.

In another embodiment of the presently disclosed subject matter, an assay method for diagnosis of an individual having a disorder associated with reduced or mutated production of the SAPAP3 protein is disclosed. In this method a tissue sample, group of cells or the like are obtained from the individual to be diagnosed. The cells are then subjected to a quantitative detection of the amount of SAPAP3 protein in the sample. This amount is then compared with the amount of functional SAPAP3 protein produced in an equivalent amount of wild type cells. The quantities of the test individual and wild type cells are compared and where a significant or substantial decrease in the SAPAP3 protein is observed by such comparison the individual is diagnosed with a clinical disorder of the SAPAP3 gene.

Tissues suitable for biopsy in the above-described method are tissues in which SAPAP3 is normally expressed. Such tissues include, without limitation, testis, muscle, and brain. In some embodiments, the biopsy is obtained from muscle or peripheral nerve tissue.

SAPAP3 expression in the biopsied tissue cells can be quantitatively detected by a number of techniques known in the art. Techniques which quantitatively detect the protein product are useful in this respect, and are generally performed by immunoassay (e.g., ELISA, protein or Western blot analysis, and immunofluorescence). Antibodies which specifically recognize the SAPAP3 protein are known in the art and available to or easily generated by the skilled practitioner. Certain immunoassay-based techniques also provide information regarding the protein product, (e.g., size and subcellular localization) which can also serve as an indicator of aberrant SAPAP3 expression or function.

Techniques that quantitatively detect the amount of SAPAP3 mRNA are also useful for such diagnosis. Such techniques are generally performed using a hybridization-based assay (e.g., RNA or Northern blot analysis, reverse transcriptase polymerase chain reaction (RT-PCR). Probes appropriate for detection of SAPAP3 mRNA in Northern blot analysis are a SAPAP3 cDNA or a portion thereof which specifically hybridizes to SAPAP3 mRNA under stringent conditions. Primers for RT-PCR analysis of SAPAP3 expression can be determined by one of skill in the art. Such assays are utilized to determine RNA size and sequence, which can also serve as an indicator of aberrant expression or protein function. These assays can also potentially cross react with a mutant SAPAP3 allele which is expressed at normal levels but is impaired in function. Therefore a negative result does not necessarily rule out the presence of a SAPAP3 disorder.

One technique for analysis of the SAPAP3 gene is PCR amplification of one or more regions of the SAPAP3 gene, followed by size analysis of the amplified product. A detectable difference in size of the amplified product, as compared to that from identically amplified wild type gene, is an indication of the presence of a mutation. A mutation which alters the size of the amplified product is likely to alter SAPAP3 expression or function, and should be further analyzed (e.g., by sequencing the amplified region). Some mutations which affect gene expression or protein function might not alter the size of the amplified region, and must be identified by other techniques, such as direct sequencing. Regions that can be targeted for amplification include, but are not limited to coding regions of the SAPAP3 gene (e.g., exons). As mutations which affect expression and function have also been known to occur in non-coding regions, examination of non-coding regions (e.g., introns) can also be performed.

Another aspect of the presently disclosed subject matter is a method for treating an individual diagnosed with a SAPAP3 disorder. Restoration of a functional SAPAP3 gene in such an individual is expected to have a therapeutic effect. Such restoration is achieved by introducing an expression vector containing a nucleic acid encoding the wild type form of the SAPAP3 protein into cells of the individual under conditions appropriate for expression.

Cells of the individual appropriate for targeted introduction of the expression vector are cells known to naturally express the wild type SAPAP3, are discussed above. In the absence of restoring wild type SAPAP3 expression to all such cells in the individual, restoration of wild type SAPAP3 expression in a subset of these cells is expected to provide significant therapeutic benefit. In some embodiments, targeted cells include, without limitation, striatum medium spiny neurons. As disclosed herein indications are that SAPAP3 expression in these cells is of particular functional significance.

Expression vectors currently known in the art and suitable for use in the above described method include, without limitation, adenovirus-based expression vectors, described in, for example U.S. Pat. Nos. 5,670,488; 5,756,086; 5,707,618; and 5,731,172; the disclosure of each of which is incorporated herein by reference in its entirety. Also included are gutted adenovirus delivery systems (Clemens et al., Gene Therapy 3: 965-972, 1996), and adeno-associated virus (AAV) based vectors, some examples of which are described in U.S. Pat. Nos. 5,858,351; 4,797,368; 5,153,414; 5,252,479; 5,354,678; and 5,756,283; the disclosure of each of which is incorporated herein by reference in its entirety. In some embodiments, the expression vector is an adenovirus vector or an adeno-associated virus vector.

The nucleic acid that encodes the SAPAP3 protein is a SAPAP3 cDNA, genomic DNA, or any combination of DNA sequences from which translation will produce wild type SAPAP3 protein. In some embodiments, the nucleic acids have a sequence derived from, or which encode a SAPAP3 protein of the species in which it is to be introduced. Significant benefit can additionally result from the inclusion of gene regulatory elements (e.g., promoter elements) that are specific for the SAPAP3 gene.

Compounds produced or identified as active compounds by application of the assay procedures described herein to the test compounds described herein are useful in vitro and in vivo as a treatment for anxiety. Subjects that can be treated by the compounds identified by the presently disclosed subject matter include both human subjects and animal subjects (e.g., dogs, cats, horses, cattle) for veterinary purposes.

Thus, as noted above, further aspects of the presently disclosed subject matter include test compounds and therapeutic agents which are active compounds produced or identified by the methods described hereinabove and pharmaceutical formulations of the same (e.g., said compound in a sterile pyrogen-free saline solution), along with the use of such compounds for the preparation of a medicament for the treatment of anxiety either alone or in combination with other compositions to treat anxiety. The formulations can be used to treat as an active compound or can be used as test compounds for the identification of active compounds.

The test and therapeutic compounds described above can be combined with a pharmaceutical carrier in accordance with known techniques to provide a pharmaceutical formulation useful carrying out the methods described above. See e.g., Remington, The Science And Practice of Pharmacy (9^{th} Ed. 1995; Mack Publishing Co., Easton, Pa.). In the manufacture of a pharmaceutical formulation according to the presently disclosed subject matter, the active compound (including the physiologically acceptable salts thereof) is typically admixed with, inter alia, an acceptable carrier. The carrier must, of course, be acceptable in the sense of being compatible with any other ingredients in the formulation and must not be deleterious to the patient. The carrier can be a solid or a liquid, or both, and in some embodiments is formulated with the compound as a unit-dose formulation, for example, a tablet, which can contain from 0.01 or 0.5% to 95% or 99% by weight of the active compound. One or more active compounds can be incorporated in the formulations of the presently disclosed subject matter, which can be prepared by any of the well known techniques of pharmacy consisting essentially of admixing the components, optionally including one or more accessory ingredients.

The formulations of the presently disclosed subject matter include those suitable for oral, rectal, topical, buccal (e.g., sub-lingual), parenteral (e.g., subcutaneous, intramuscular, intradermal, or intravenous), topical (i.e., both skin and mucosal surfaces), the most suitable route in any given case will depend on the nature and severity of the condition being treated and on the nature of the particular active compound which is being used.

Formulations of the presently disclosed subject matter suitable for parenteral administration comprise sterile aqueous and non-aqueous injection solutions of the active compound, which preparations are in some embodiments isotonic with the blood of the intended recipient. These preparations can contain anti-oxidants, buffers, bacteriostats and solutes which render the formulation isotonic with the blood of the intended recipient. Aqueous and non-aqueous sterile suspensions can include suspending agents and thickening agents. The formulations can be presented in unit-dose or multi-dose containers, for example sealed ampoules and vials, and can be stored in a freeze-dried (lyophilized) condition requiring only the addition of the sterile liquid carrier, for example, saline or water-for-injection immediately prior to use. Extemporaneous injection solutions and suspensions can be prepared from sterile powders, granules, and tablets of the kind previously described. For example, in one aspect of the presently disclosed subject matter, there is provided an injectable, stable, sterile composition, or a salt thereof, in a unit dosage form in a sealed container. The compound or salt is provided in the form of a lyophilizate which is capable of being reconstituted with a suitable pharmaceutically acceptable carrier to form a liquid composition suitable for injection thereof into a subject.

The unit dosage form typically comprises from about 10 mg to about 10 grams of the compound or salt. When the compound or salt is substantially water-insoluble, a sufficient amount of emulsifying agent which is physiologically acceptable can be employed in sufficient quantity to emulsify the compound or salt in an aqueous carrier.

Formulations suitable for topical application to the skin can in some embodiments take the form of an ointment, cream, lotion, paste, gel, spray, aerosol, or oil. Carriers which can be used include petroleum jelly, lanoline, polyethylene glycols, alcohols, transdermal enhancers, and combinations of two or more thereof.

Formulations suitable for transdermal administration can be presented as discrete patches adapted to remain in intimate contact with the epidermis of the recipient for a prolonged period of time. Formulations suitable for transdermal administration can also be delivered by iontophoresis (see e.g., Tyle, Pharmaceutical Research 3:318-326, 1986) and typically take the form of an optionally buffered aqueous solution of the active compound. Suitable formulations comprise citrate or bistris buffer (pH 6) or ethanol/water and contain from 0.1 to 0.2 M active ingredient.

Formulations suitable for oral administration may be presented in discrete units, such as capsules, cachets, lozenges, or tablets, each containing a predetermined amount of the active compound; as a powder or granules; as a solution or a suspension in an aqueous or non-aqueous liquid; or as an oil-in-water or water-in-oil emulsion. Such formulations may be prepared by any suitable method of pharmacy which includes the step of bringing into association the active compound and a suitable carrier (which may contain one or more accessory ingredients as noted above). In general, the formulations of the presently disclosed subject matter are prepared by uniformly and intimately admixing the active compound with a liquid or finely divided solid carrier, or both, and then, if necessary, shaping the resulting mixture. For example, a tablet may be prepared by compressing or molding a powder or granules containing the active compound, optionally with one or more accessory ingredients. Compressed tablets may be prepared by compressing, in a suitable machine, the compound in a free-flowing form, such as a powder or granules optionally mixed with a binder, lubricant, inert diluent, and/or surface active/dispersing agent(s). Molded tablets may be made by molding, in a suitable machine, the powdered compound moistened with an inert liquid binder.

### EXAMPLES

The following Examples have been included to illustrate modes of the presently disclosed subject matter. In light of the present disclosure and the general level of skill in the art, those of skill will appreciate that the following Examples are intended to be exemplary only and that numerous changes, modifications, and alterations can be employed without departing from the scope of the presently disclosed subject matter.

### Example 1

### Production of SAPAP3 Knockout Mice

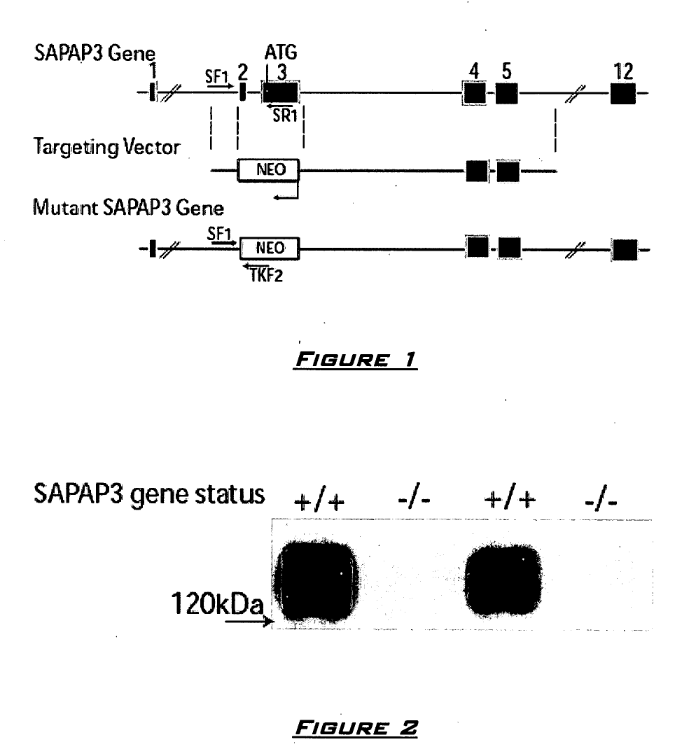

A targeting vector was constructed such that sections of SAPAP3 mouse genomic DNA flanking exon 2 (containing the methionine start codon) and exon 3 were cloned and inserted on either side of a conventional Neomycin resistance cassette using standard methods: PCR cloning from genomic DNA, restriction enzyme cutting, and ligation into the base vector (see FIG. 1). The vector also contained a standard thymidine kinase cassette following the section of genomic DNA flanking exon 3 in the base vector.

The targeting vector was linearized with Not I, purified, and electroporated into R1 mouse embryonic stem cells using standard methods (see e.g., Hogan et al. Manipulating the Mouse Embryo: A Laboratory Manual, 2nd ed., Cold Spring Harbor Laboratory Press, Cold Spring Harbor, N.Y., 1994). Transfected colonies resistant to both G418 and gancyclovir were screened by PCR and Southern blot to confirm heterozygous replacement of SAPAP3 exons 2 and 3 with the Neomycin cassette. One colony so positively confirmed was expanded in cell culture and injected in C57BL/6 blastocysts using standard methods. Chimeric male pups were obtained and mated with C57BL/6 female; germline transmission into offspring was confirmed by PCR. Heterozygous mice were crossed to obtain homozygous mice.

The biochemical fractions most concentrated in SAPAP3 protein, the PSDII/III fractions (see Cho et al. Neuron 9:929-42, 1992; Welch et al. J Comp Neurol 472:24-39, 2004) were prepared from wild type littermate and homozygous mice. No SAPAP3 protein was detected in PSD II or III fractions in mice homozygous for interruption of exons 2 and 3 (see FIG. 2). All experiments were conducted in accordance with the NIH guidelines for the care and use of animals.

### Example 2

### Identification of Lesions on SAPAP3 Knockout Mice and Recognition of Compulsive Grooming as the Origin of the Lesions

SAPAP3 knockout mice were viable and able to mate to produce homozygous knockout offspring. However, SAPAP3 knockout mice began to develop obvious facial lesions between 4-12 months of age.

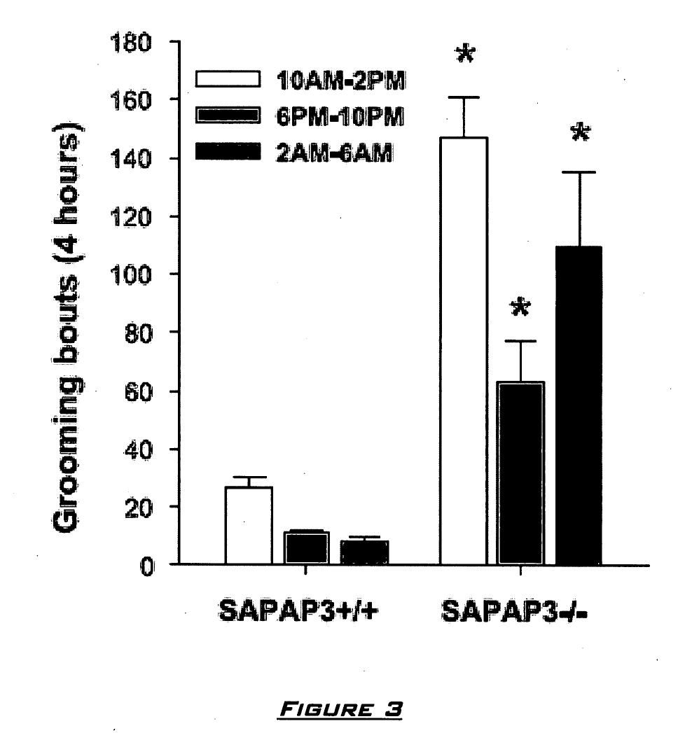

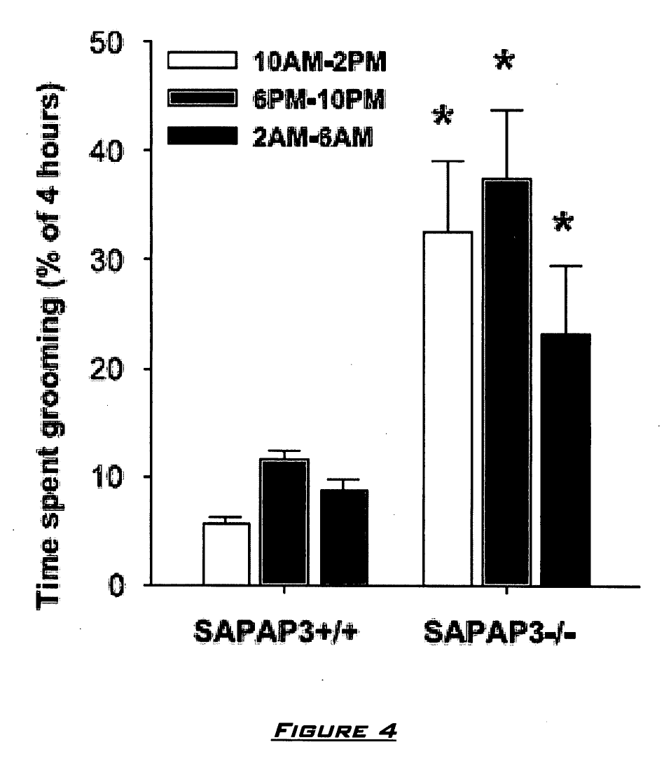

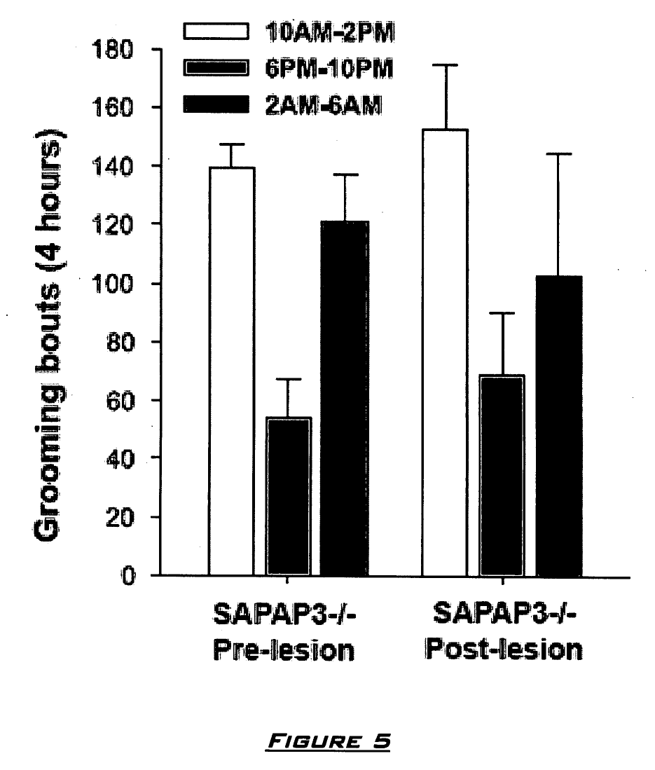

To ascertain the source of the lesions quantitative, video studies of the knockout mice were performed. Groups of 10 wild type mice and 10 knockout mice were housed such that each mouse was isolated in a single cage. Following an acclimatization period to individual housing, each mouse was videotaped for 24 hours. Three, four-hour sections (10 AM-2 PM, 6 PM-10 PM, and 2 AM-6 AM) of each 24-hour period were watched and scored for a panel of behaviors: grooming, drinking/eating, sleeping, rearing, digging/shifting, or alert/active. When analyzed on either a “time spent” or “number of bouts engaged in” basis, it was obvious that SAPAP3 knockout mice groomed far more than their wild type counterparts in every time period examined (see FIGS. 3 and 4). Additionally, in comparing grooming behavior within pre-lesion or post-lesion groups of SAPAP3 knockout mice, no difference in grooming behavior was found, demonstrating that the lesions themselves were not the primary cause of excess grooming (see FIG. 5).

### Example 3

### Identification of Innate Anxiety Accompanying Compulsive Behavior in SAPAP3 Knockout Mice

The compulsive behavior of the mice as identified in Example 2 appeared to be reminiscent of human Obsessive Compulsive Disorder (OCD). OCD is usually accompanied by abnormally high anxiety in humans, and the SAPAP3 mice were therefore tested for overly anxious behavior. SAPAP3 mice and wild type controls on the same genetic background were tested in three different paradigms designed to reveal anxious behavior: 1) the “open field” test, 2) the “light-dark box test, and 3) the “elevated zero maze” test. SAPAP3 mice exhibited a statistically significant tendency toward anxiety on all three tests in comparison to wild type controls.

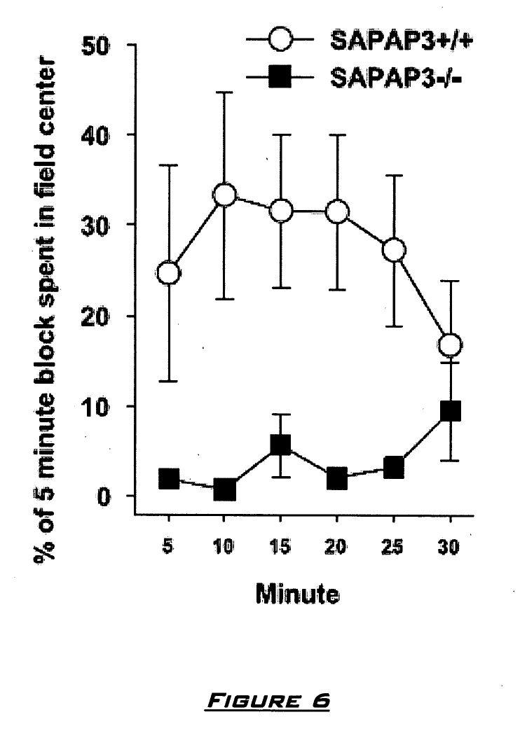

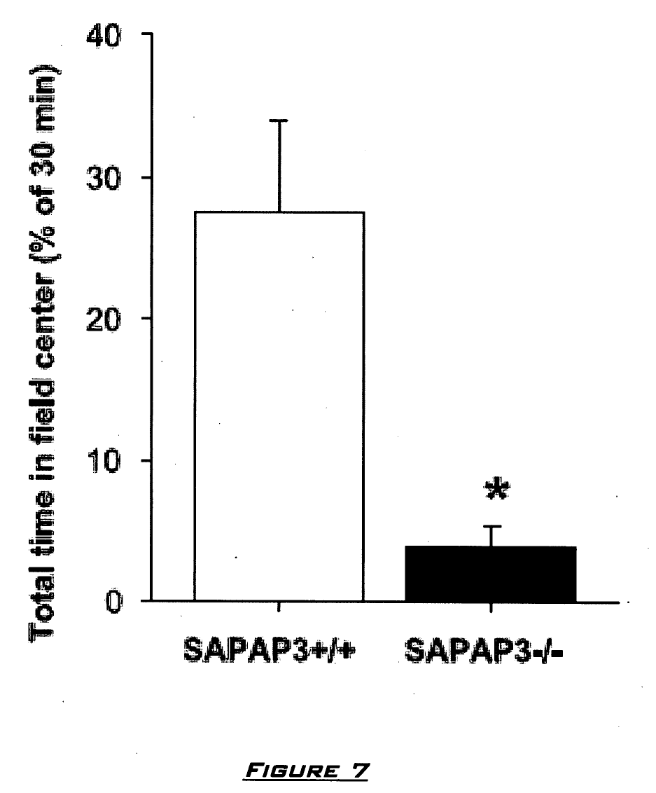

In the open field test, mice were placed into an “open field” of rodent bedding with walls enclosing the open space. In the open field test, the wild type mice spent much less time or distance traveled exploring the center of the open field (a riskier environment than the edge of the field near the wall; see FIGS. 6 and 7). Total distance traveled at the periphery (thigmotaxis), however, was not statistically different between the genotypes—indicating that the SAPAP3 knockout mice did not have a motor problem or spend an excessive amount of time grooming.

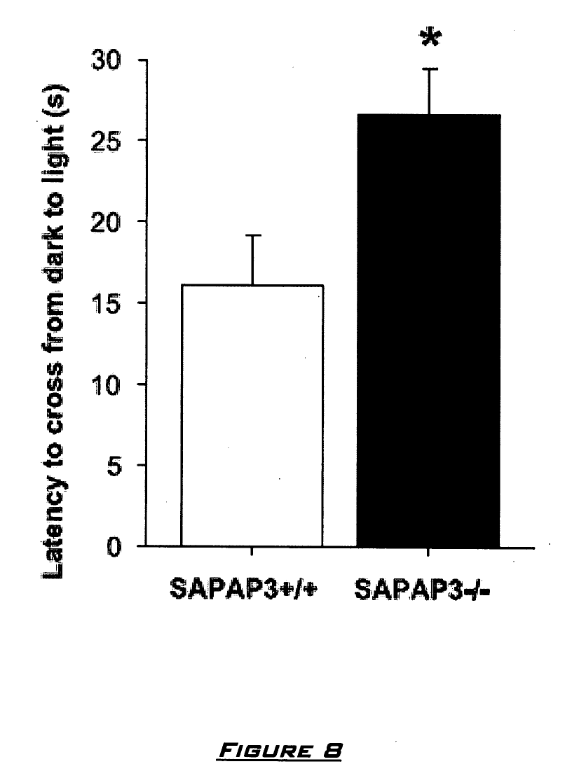

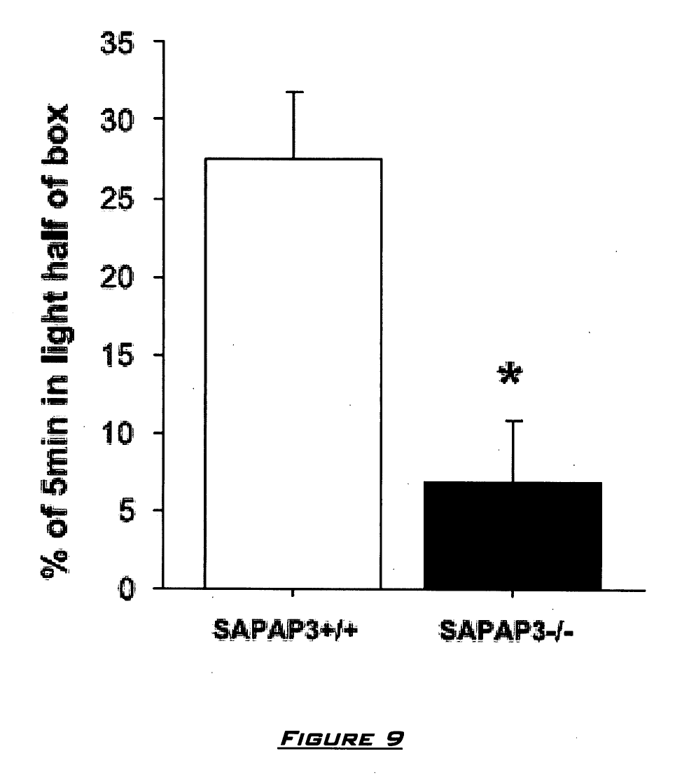

In the light dark box test, SAPAP3 knockout mice and wild type mice were individually placed into the dark half a two chamber box. The box contained a completely dark chamber and a chamber lit with a bright light. At time=0, the door was opened between the two chambers. On average, the SAPAP3 knockout mice took statistically longer to begin exploring the lit chamber and the SAPAP3 knockout mice spent statistically less time in the lit chamber, both indications of anxious behavior in the SAPAP3 knockout mice (see FIGS. 8 and 9.)

In the elevated zero maze test, SAPAP3 knockout or wild type mice were individually placed onto a circular platform approximately 24 inches off of the ground. The section of the platform onto which the mice were placed had walls, ensuring that the mice could not fall off. 50% of the platform contained no walls. On average, the SAPAP3 knockout mice spent less time on the open portion of the platform and crossed from the walled to open areas fewer times than wild type mice. Both of these observations were indications that the SAPAP3 knockout mice were innately anxious.

### Example 4

### SAPAP3 Knockout Mice did not Contain Obvious Peripheral Defects in Addition to Central Compulsive and Anxiety Defects Described in Preceding Examples

The most commonly lesioned site on SAPAP3 knockout mice was the skin just below the eye. Lesions usually began there and progressed. Skin from just below the eye was taken from pre-lesion SAPAP3 knockout mice and wild type mice and processed for histological observation using standard hematoxylin and eosin staining. No difference was observed between the genotypes.

SAPAP3 knockout mice were bread onto the GFPJ line of mice (Feng et al. Neuron 28:41-51 2000). Skin from just below the eye of SAPAP3 knockout GFPJ mice and wild type GFPJ mice was taken and processed for fluorescent confocal microscopy. No difference in the structure of the peripheral nervous system was evident in comparing the two genotypes.

### Example 5

### Treatment of the SAPAP3 Knockout Mice with Fluoxetine Improved Compulsive Overgrooming, Anxiety, and Lesions

Two cohorts of eight SAPAP3 knockout mice and two cohorts of eight wild type mice were housed in isolation and allowed to acclimate as in Example 2. One cohort of each genotype served as an experimental group and the other as a vehicle control group. The experimental groups were given a daily intraperitoneal injection of 5 mg/kg Fluoxetine for fourteen days. The control groups were given a daily vehicle injection under the same conditions.

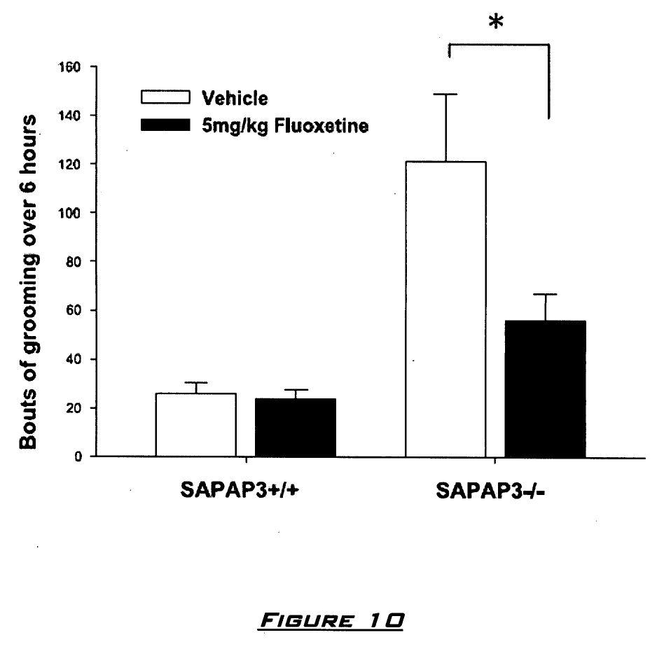

After six days, mice from each group were individually videotaped and their behaviors scored as in Example 2. In comparing the SAPAP3 knockout Fluoxetine group with the SAPAP3 knockout vehicle control group, it was evident that the Fluoxetine group engaged in far fewer bouts of grooming than did the vehicle group. Fluoxetine was therefore efficacious in treating the compulsive overgrooming of the SAPAP3 knockout mice (see FIG. 10).

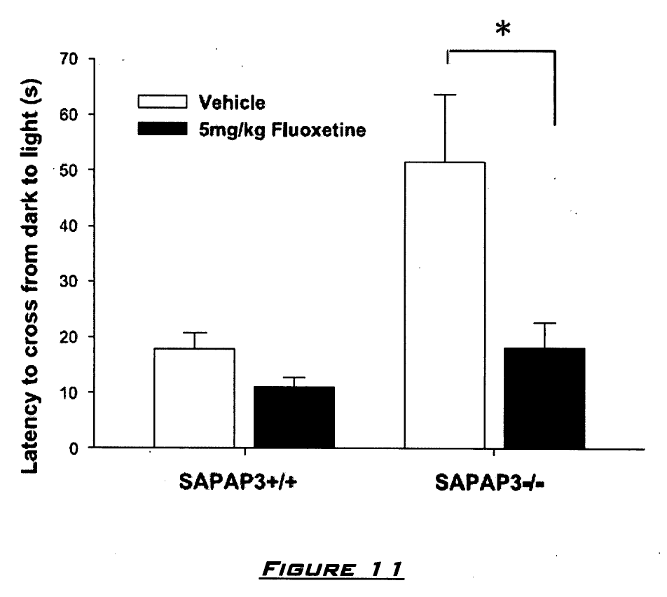

Following videotaping, the four groups of mice were tested on the light-dark box emergence test. The performance of the SAPAP3 knockout mice was significantly improved after 6 days of treatment with 5 mg/kg Fluoxetine (see FIG. 11).

Four cohorts of eight SAPAP3 knockout mice and four cohorts of eight wild type mice were housed in isolation and allowed to acclimate as above. One cohort of each genotype served as a 5 mg/kg Fluoxetine experimental group, one as a 10 mg/kg Fluoxetine experimental group, one as a vehicle control group, and one as an uninjected control group. Based on a semi-quantitative scoring of lesion severity (on a scale of 0-4, with a higher number representing a more severe lesion), Fluoxetine stabilized or improved lesions over a 2 week period (Fluoxetine groups considered on a combined basis vs. vehicle and uninjected groups considered on a combined basis: see Table 1).

TABLE 1
Fluoxetine Treatment Reduces Lesion Severity in SAP3 KO Mice

| | | No | |
|---|---|---|---|
| | Worse | Change | Improve |
| Vehicle/Untreated | 18.8% | 75% | 6.2% |
| N = 16 | | | |
| Fluoxetine | 0% | 71.4% | 28.6% |
| N = 16 | | | |

N = 32 mice tested at two weeks
X² _{vehicle} (2, 29) = 4.571, p < 0.102 (2-tail)
X² _{Fluoxetine} (2, 29) = 5.275, p < 0.022 (2-tail)

### Example 6

### Rescue of the Defect Observed in SAPAP3 Knockout Mice using Transgenic Expression of SAPAP3 in the Striatum

20 SAPAP3 knockout mice were injected with lentivirus in the dorsal region of the striatum at 1 week of age. 11 of the mice were injected with virus expressing the coding region of SAPAP3 and 9 of the mice were injected with a control virus. The mice were allowed to develop for 3-9 months and were then videotaped under the isolated conditions described in Examples 2 and 5. Analysis of the videotape revealed that SAPAP3 knockout mice injected with experimental virus encoding SAPAP3 engaged in significantly fewer grooming bouts than SAPAP3 mice injected with control virus. Thus, the defect observed in the SAPAP3 knockout mice can be rescued by specific expression of SAPAP3 in the striatum starting at 1 week of age.

Analysis of the videotape revealed that SAPAP3 knockout mice injected with experimental virus encoding SAPAP3 engaged in significantly fewer grooming bouts than SAPAP3 mice injected with control virus. Thus, the defect observed in the SAPAP3 knockout mice was rescued by transgenic expression of SAPAP3 in the striatum starting at 1 week of age.

### Example 7

### SAPAP3 Knockout Mice have a Defect in Overall Cortico-striatal Glutamatergic Input

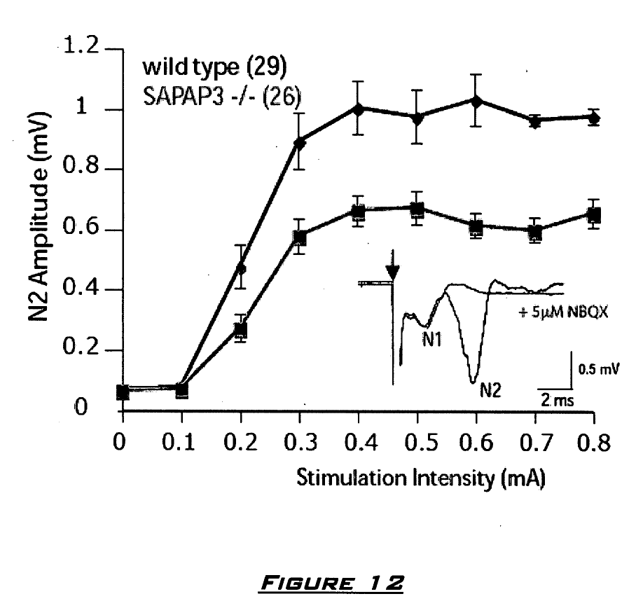

SAPAP3 knockout mice and wild type controls were sacrificed and brain slices were cut such that cortico-striatal connections were maintained. Slices were analyzed individually. A stimulating electrode was placed in the ventral corpus collosum and an extracellular recording electrode was placed in the dorsal striatum. An input-output relationship between the stimulation of cortico-striatal fibers and striatal population EPSPs was developed using graded stimulation. It was statistically clear that cortico-striatal connections were weaker in slices from SAPAP3 knockout mice than in WT control slices (i.e. the plateau of the input-output curve was lower—see FIG. 12).

### Example 8

### SAPAP3 Knockout Mice have an Overall Increase in NMDA Receptor Current at the Cortico-striatal Synapse, with a Selective Upregulation of NR2a

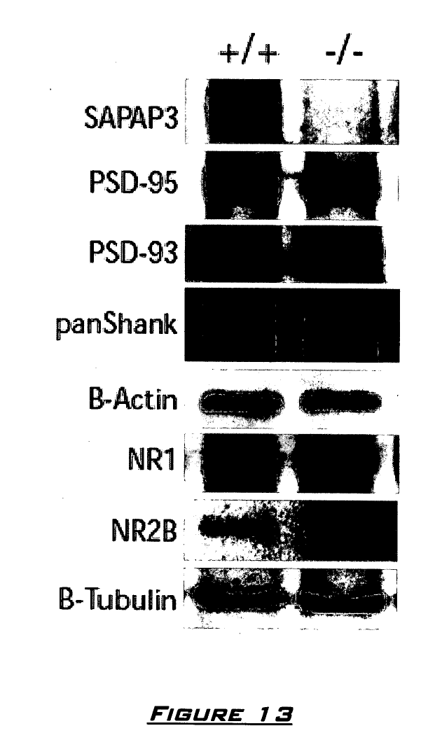

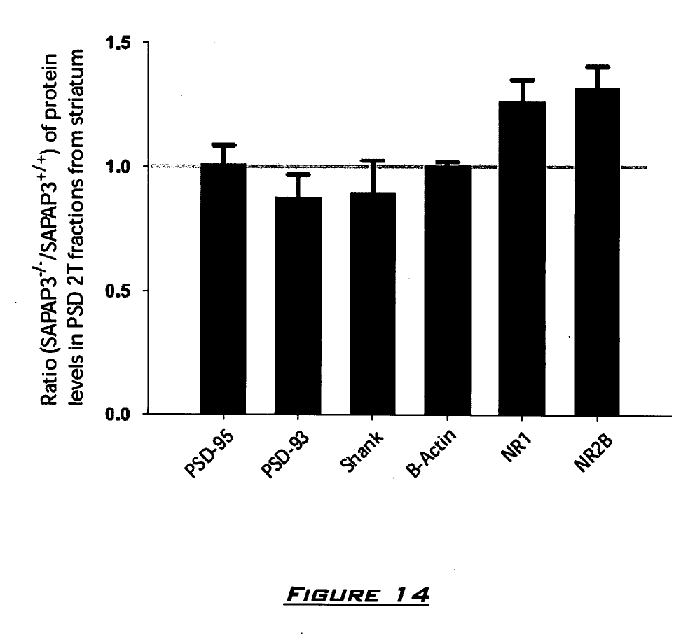

Three pairs of SAPAP3 knockout and wild type control mice were used to generate 3 paired preparations of post-synaptic density fractions through the standard PSD biochemical fractionation method (Cho et al. Neuron 9:929-942, 1992). These paired samples were probed with antibodies to several proteins known to be present in the post-synaptic density, including receptors, scaffolding proteins, and cytoskeletal elements. Protein levels were normalized to levels of β-tubulin, which were determined to be identical between SAPAP3 knockout and wild type mice. All proteins were determined to be of statistically equal levels between SAPAP3 knockout and wild type mice, with the very important exception of NMDA receptor subunits. The NR1 and NR2B NMDA receptor subunits were found to be approximately 30% overly abundant in the PSD fractions from SAPAP3 knockout mice, and the NR2A receptor subunit was found to be approximately 40% under-represented in the PSD fractions from SAPAP3 knockout mice (see FIGS. 13 and 14). These observations suggest that cortico-striatal connections in SAPAP3 knockout mice are not maturing through the normal developmental stage in which NR1 and NR2B expression are ramped down and NR2A expression is ramped up (van Zundert et al. Trends in Neurosciences 27:428-37, 2004).

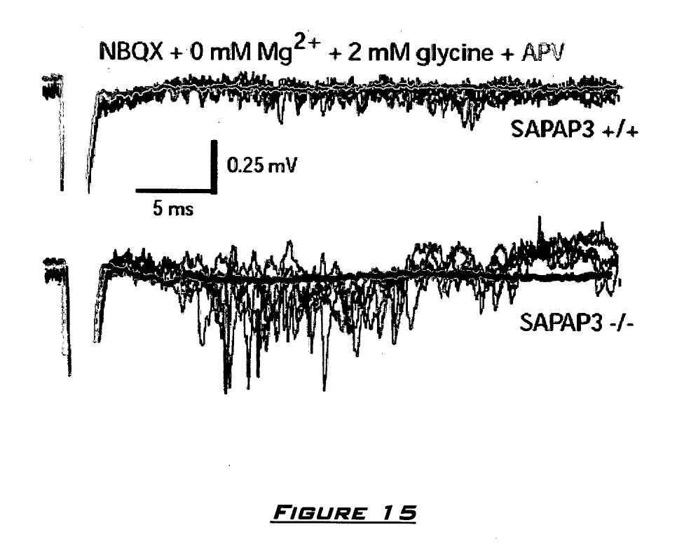

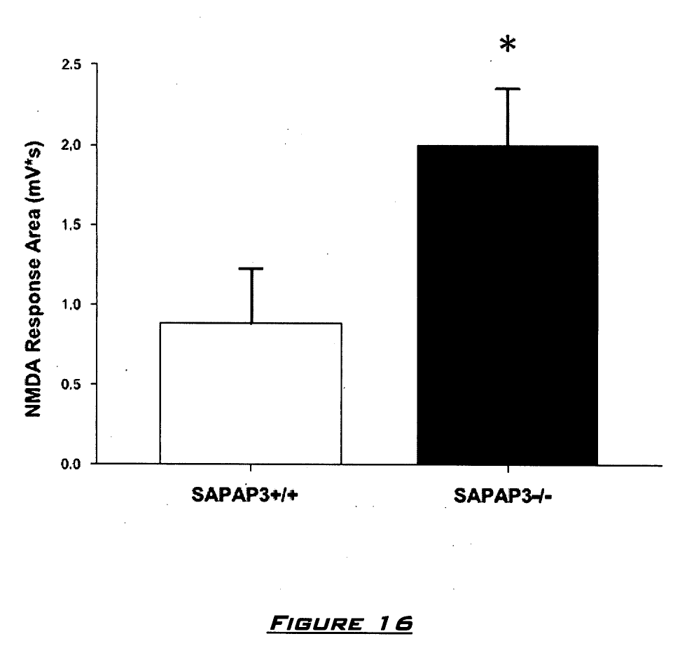

Additionally, brain slices were analyzed electrophysiologically as described in Example 7, except that the AMPA receptor blocker CNQX was present in the bath, thereby isolating NMDA receptor currents. Under this paradigm, it was found that equal levels of stimulation resulted in correspondingly more NMDA receptor current at cortico-striatal synapses in slices from SAPAP3 knockout mice than in slices from wild type mice (see FIGS. 15 and 16).

### Example 9

### SAPAP3 Mice Have Innate Serotonin Imbalance in Several Brain Regions

SAPAP3 knockout mice and wild type mice were sacrificed and the frontal cortex, ventral striatum, and dorsal striatum were carefully dissected. Mono-amine neurotransmitters were biochemically extracted and levels of each transmitter in each brain region from each mouse were analyzed using high pressure liquid chromatography. Serotonin levels were found to be altered in both the cortex and the striatum. Additionally, it was found that high doses of Fluoxetine (10 mg/kg and above) were progressively more toxic to SAPAP3 knockout animals than to wild type controls animals—an additional suggestion that serotonin imbalances exist in the SAPAP3 knockout animals.

### Example 10

### SAPAP3 Mice are Used to Screen to Small Molecules for Efficacy in Treating Anxiety Disorders

Using exact dosing through intraperitoneal injections as in Example 5, SAPAP3 knockout experimental mice and wild type controls are dosed with experimental compounds. Following 3-30 days of dosing, or thereabouts, both groups of mice are tested using the videotaping studies described in Example 2. The mice are also tested for anxiety as described in Example 3. In this fashion, the SAPAP3 knockout mice function as a screen for compounds likely to be active in treating anxiety disorders in humans.

Simultaneously, toxic side effects of the same set of compounds are assayed.

Using this manner of testing, efficacious, non-toxic compounds are identified for testing in human clinical trials as treatments for anxiety disorders.

### Example 11

### Use of the SAPAP3 Protein as a Therapeutic Target for Developing Novel Anti-anxiolytic Compositions

As demonstrated in Examples 7 and 8, the presence or absence of SAPAP3 can have profound effects on the physiology and biochemistry of the cortico-striatal synapse. These effects will alter the dynamics of circuitry interconnecting the cortex, basal ganglia, and thalamus (also known as the “OCD circuit”).

Several families of proteins are known to interact with SAPAP3, including the MAGUK (membrane associated guanylate kinase) family (e.g., PSD-95), the SHANK family, kinases, kinase binding proteins, and others. Blocking or facilitating any of these interaction sites on the SAPAP3 protein with an appropriate small molecule, followed by behavioral testing as noted in Examples 2, 5, and 10 above, allows an assay of whether a particular molecule and/or SAPAP3 interaction site generate the desired behavioral outcome.

### Example 12

### Use of SAPAP3 Gene Status as a Genetic Test of Propensity to Develop Anxiety Disorders and/or as a Test for Likely Efficacy Response of Particular Classes of Anti-anxiolytic Compositions or Other Psychiatric Medication

Screening of SAPAP3 gene status in appropriate populations of patients (e.g., family members with anxiety disorders) in comparison to appropriate controls (family members without anxiety disorders) reveals whether or not SAPAP3 is frequently mutated in patients with anxiety disorders. SAPAP3 is frequently mutated in patients with anxiety disorders, so a test for SAPAP3 status will reveal the propensity for an individual to develop anxiety disorders.

Similarly, SAPAP3 status is used to predict the likely efficacy of certain classes of psychiatric therapeutics, such as selective serotonin reuptake inhibitors. This possibility is evident, given the serotonin and other neurochemistry defects in the SAPAP3 knockout mice, and would certainly be true for any future therapeutics designed to target SAPAP3 or other proteins (such as NMDA subunits) in the post-synaptic density pathway in which SAPAP3 participates.

### Example 13

### Use of the SAPAP3 Gene as a Treatment of Anxiety Disorders

Using a brain gene-therapy approach, the SAPAP3 gene itself is used as a treatment for anxiety disorders. The SAPAP3 gene is delivered to striatal medium spiny neurons through a viral vector or other approach and inserted into the genomic DNA of those cells, allowing them to express SAPAP3.

It will be understood that various details of the presently disclosed subject matter may be changed without departing from the scope of the presently disclosed subject matter. Furthermore, the foregoing description is for the purpose of illustration only, and not for the purpose of limitation.

What is claimed is:

1. A transgenic non-human mammal, wherein the transgenic non-human mammal comprises a disruption in one or both alleles of an endogenous SAPAP3 gene, and further wherein the disruption results in the non-human mammal exhibiting increased levels of anxiety as compared to a wild type non-human mammal of the same species that does not comprise a disruption in one or both alleles of its endogenous SAPAP3 gene.

2. The transgenic non-human mammal of claim 1, wherein the transgenic non-human mammal is a mouse.

3. The transgenic non-human mammal of claim 1, wherein the disruption results from a targeted deletion of a portion of the endogenous SAPAP3 gene.

4. The transgenic non-human mammal of claim 1, wherein the deleted portion of the endogenous SAPAP3 gene is replaced with a nucleotide sequence encoding an antibiotic resistance polypeptide.

5. The transgenic non-human mammal of claim 1, wherein the disruption results from a deletion of the second and third exons of the SAPAP3 gene.

6. The transgenic non-human mammal of claim 1, wherein the disruption prevents the expression of a functional SAPAP3 protein in cells of the transgenic non-human mammal.

7. The transgenic non-human mammal claim 1, wherein the transgenic non-human mammal comprises one or more disruptions of both alleles of its endogenous SAPAP3 gene.

8. A method for identifying a therapeutic agent for the treatment of anxiety in a subject, the method comprising:
(a) providing a mammalian cell comprising a disruption in an endogenous SAPAP3 gene, wherein the disruption results in a reduced level of an SAPAP3 biological activity in the mammalian cell as compared to that seen in a wild type cell under identical conditions;
(b) administering a test compound to the cell of step (a); and
(c) assaying the therapeutic effects of the test compound by comparing the level of an SAPAP3 biological activity in the cell after the administering step with the level of an SAPAP3 biological activity in the cell prior to the administering step,
wherein an increase in the level of an SAPAP3 biological activity in the cell after the administering step is an indication that the test compound is a therapeutic agent for the treatment of anxiety.

9. The method of claim 8, wherein the mammalian cell is present within a transgenic non-human mammal.

10. The method of claim 8, wherein the administering results in an upregulation in expression of an endogenous SAPAP3 allele sufficient to result in an increased level of an SAPAP3 biological activity in the cell after the administering step.

11. A method for identifying a therapeutic agent for treatment of anxiety in a subject, the method comprising:
(a) providing a non-human mammal that comprises a disruption of one or both SAPAP3 alleles, wherein the non-human mammal expresses an anxiety phenotype;
(b) administering a test compound to the non-human mammal; and
(c) comparing an anxiety phenotype in the non-human mammal before administering the test compound to an anxiety phenotype in the non-human mammal after administering the test compound,
whereby a therapeutic agent for the treatment of anxiety in a subject is identified.

12. The method of claim 11, wherein the non-human mammal is a mouse comprising one or more disruptions of both endogenous SAPAP3 alleles.

13. The method of claim 12, wherein the one or more disruptions comprise at least one targeted disruption of an SAPAP3 allele.

14. A method for diagnosing an individual with a clinical disorder associated with reduced expression of SAPAP3 comprising:
(a) providing a tissue sample from the individual;
(b) quantitatively detecting the expression of SAPAP3 protein in cells of the sample; and
(c) assaying the amount of SAPAP3 protein produced by comparing the quantity of SAPAP3 protein produced in step (b) with the quantity of SAPAP3 protein produced by cells known to exhibit the normal expression of SAPAP3 wherein a substantial decrease in SAPAP3 protein in the sample by comparison is indicative of the clinical disorder in the individual.

15. The method of claim 14, wherein the individual is a human.

16. A method for treating an individual with a clinical anxiety disorder associated with a reduced level of a biological activity of an SAPAP3 gene product in a biological sample, the method comprising;
(a) assaying a biological sample isolated from the individual to determine that the individual has a reduced level of a biological activity of an SAPAP3 gene product in the biological sample compared to the biological sample isolated from a control individual; and
(b) administering to the individual an effective amount of an enhancer of a biological activity of an SAPAP3 gene product sufficient to ameliorate the clinical anxiety disorder.

17. The method of claim 16, wherein the biological sample is selected from the group consisting of a cell, a tissue, and a fluid that normally would be expected to express an SAPAP3 gene product in an individual of the same species as the individual from which the biological sample was isolated.

18. The method of claim 16, wherein the individual is a mammal.

19. The method of claim 16, wherein the administering comprises introducing into a cell or a tissue of the individual an expression construct encoding an SAPAP3 gene product operably linked to a promoter that is active in the cell or tissue, whereby an amount of an SAPAP3 protein sufficient to ameliorate the clinical anxiety disorder is produced in the cell of the tissue.

20. The method of claim 16, wherein the enhancer of a biological activity of an SAPAP3 gene product comprises a purified SAPAP3 protein and the administering comprises administering to the individual an amount of the purified SAPAP3 protein sufficient to ameliorate the clinical anxiety disorder to the individual.
